(19) United States
(12) Patent Application Publication
(10) Pub. No.: US 2001/0051720 A1
(43) Pub. Date: Dec. 13, 2001

(54) 1,3,5-triazine-2, 4,6-hydroxyalkyl acids and derivatives

(76) Inventor: John J. Waldmann, Marblerock, CT (US)

Correspondence Address: Isaac A. Angres, 2001 Jefferson Davis Highway - Suite 301, Arlington, VA, 22202, US

(21) Appl. No.: 09/902,632

(22) Filed: Jul. 12, 2001

Related U.S. Application Data
(63) Continuation of application No. 09/408,833, filed on Sep. 30, 1999.

(51) Int. Cl. C07D 253/07

(52) U.S. Cl. 544/195; 544/196

(57) ABSTRACT

The present invention describes 1,3,5-Triazine-2,4,6-tris-hydroxyalkyl acids of the general formula I

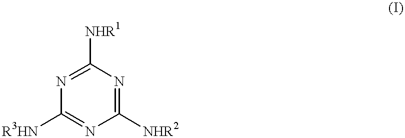

in which R¹ represents a residue of the general formula II

R′[CH(OH)—CH(OH)]ₙ (II)

in which R′ can be an acid group selected from the group consisting of carboxyl, sulfamic acid group, sulfonic acid group, and phosphate acid group, n denotes a number in the range from 1 to 10, and R² and R³ can be the same or different and represent one of the radicals R¹.

### BACKGROUND OF THE INVENTION

1. Field of the Invention

The present invention is directed to new 1,3,5-Triazine-2,4,6-hydroxyalkyl acid monomeric, oligomeric or polymeric derivatives and a process for their preparation by reacting melamine with an excess of glyoxal thereafter treating resultant mixture with a corresponding organic or mineral acid.

2. Description of the Prior Art

It is known to react melamine with aliphatic aldehyde in order to obtain N,N′,N″-tris(hydroxyalkyl)-melamine.

U.S. Pat. No. 3,806,508 discloses that tris-alkylolamino triazine-ethers can be prepared from aminotriazines, aldehydes with lower alcohols in a one-stage process.

U.S. Pat. No. 4,408,045 provides a process for the preparation of a partially etherefied monomeric methylolmelamine, which comprises carrying out a single stage addition reaction between formaldehyde and melamine, and a simultaneous etherification reaction with methanol or ethanol.

U.S. Pat. Nos. 4,618,676 and 4,668,785 disclose N,N′,N″-tris(hydroxyalkyl)-melamine and a process for their preparation by reacting melamine with alkanolamine in the presence of an acidic catalyst.

### SUMMARY OF THE INVENTION

The present invention relates to new 1,3,5-triazine-2,4,6-hydroxyalkyl acid monomeric, oligomeric or polymeric derivatives corresponding to the following general formula (I)

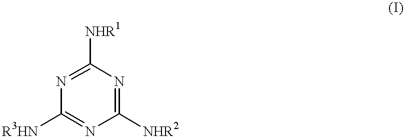

in which R¹ represents a residue of the general formula (II)

R′[CH(OH)—CH(OH)]ₙ (II)

in which R′ can be an acid group selected from the group consisting of carboxyl, sulfamic acid group, sulfonic acid group, and phosphoric acid group, n denotes a number in the range from 1 to 10,

R² and R³ can be the same or different and represent one of the radicals R¹.

The present invention also relates to a process for the preparation of 1,3,5-triazine-2,4,6-hydroxyalkyl acid monomeric, oligomeric or polymeric derivatives according to the invention, characterized in that reacting melamine with glyoxal in the presence of an acid.

### DETAILED DESCRIPTION OF THE INVENTION

The process of the invention is carried out by mixing melamine and glyoxal solution at a temperature ranging from 60° C. to 80° C., while stirring for 10 to 30 minutes. Then the mixture is cooled down to 55-60° C., an acid is added. The entire mixture is heated to 60-70° C. and maintained at this temperature for 20 to 30 minutes. The product is cooled down to room temperature for recovery and purification.

The acid to be used in the process according to this invention may be organic acid or mineral acid. Suitable organic acids include aliphatic monocarboxylic acid such as formic acid, acetic acid, propionic acid, and the like; polycarboxylic acid such as citric acid; hydroxycarboxylic acid such as glyoxylic acid. Suitable mineral acids include sulfamic acid, sulfuric acid, phosphoric acid and hydrochloric acid.

The molar ratio of glyoxal to melamine is from 3:1 to 4:1. The use of great excess of glyoxal has no further advantages since in this case the reaction time is increased considerably.

The molar ratio of acid to melamine is from 3:1 to 3.5:1. The excess acid can be neutralized with ammonia organic or inorganic base.

The preferred melamine:glyoxal:acid ratio is 1:3:3.05. With respect to the reaction of melamine with glyoxal, the preferred reaction temperature is from 65° C. to 70° C. , the preferred reaction time is from 10 to 20 minutes. The preferred acid treatment temperature is from 60° C. to 65° C., the preferred acid treatment time is around 20 minutes.

The preferred 1,3,5-triazine-2,4,6-hydroxyalkyl acid derivatives according to this invention are those corresponding to the above general formula (I) in which R¹, R² and R³ represent the same radicals of the general formula (II), in which n denotes the number 1 and R′ represents carboxyl group.

1,3,5-Triazine-2,4,6-hydroxyalkyl acid derivatives according to this invention are obtained in liquid form and in high yields and are recovered by neutralization of excess acid, filtration and evaporation of the solvent. Distillation under high vacuum provides exceptionally pure products.

The new 1,3,5-triazine-2,4,6-hydroxyalkyl acid derivatives according to this invention are suitable for use as odor suppressants in waste water treatment, as additives for flame retardants, or as intermediates in the manufacture of resins and dyestuffs.

The invention is further illustrated but is not intended to be limited by the following examples in which all parts and percentage are by weight unless otherwise specified.

### EXAMPLES

### Example 1

In a one liter, three-necked, round-bottomed flask provided with a thermometer, a reflux condenser, and stirrer were prepared 116 parts by weight (pbw) of glyoxal (0.6 mole) solution and heated to 70° C. 25.2 parts by weight (pbw) of melamine (0.2 mole) were added to the above glyoxal solution with stirring, and reacted for ten minutes. The mixture was cooled to 55 to 60° C. by adding to the reactor 47 parts by weight of distilled water. To the mixture were added, with stirring, 58.20 parts by weight of sulfamic acid (0.606 mole) and the temperature was maintained at 55 to 60° C. The entire mixture was heated and kept the temperature at 60 to 65° C. for 20 minutes. The product is cooled down to room temperature for recovery. A brown liquid solution of 1,3,5-triazine-2,4,6-hydroxyalkyl trisulfamic acid is formed.

### Example 2

The procedures as described in Example 1 were repeated except that 88.80 grams (0.60 mole) of glyoxylic acid (50%) were employed in place of sulfamic acid. The entire mixture was heated to 60-65° C. and kept at this temperature for 20 minutes, then cooled down to room temperature. A yellow fluorescent liquid is formed, which is 1,3,5-triazine-2,4,6-hydroxyalkly tricarboxylic acid.

### Example 3

The procedures as described in Example 1 were repeated except that 0.60 mole of phosphoric acid was employed in place of sulfamic acid. The entire mixture was heated to 60-65° C. and kept at this temperature for 20 minutes, then cooled down to room temperature. A yellow liquid is obtained, which is 1,3,5-triazine-2,4,6-hydroxyalkly triphosphoric acid.

### Example 4

The procedures as described in Example 1 were repeated except that 0.60 mole of sulfuric acid was employed in place of sulfamic acid. The entire mixture was heated to 60-65° C. and kept at this temperature for 20 minutes, then cooled down to room temperature. A yellow liquid is obtained, which is 1,3,5-triazine-2,4,6-hydroxyalkly trisulfonic acid.

It is to be understood that the forms of the invention herewith described are to be taken as preferred examples of the same, and that various changes may be resorted to, without departing from the spirit of the invention or the scope of the subjoined claims.

I claim:

1. 1,3,5-Triazine-2,4,6-tris-hydroxyalkyl acids of the general formula I 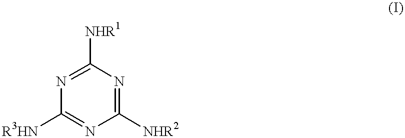
in which R¹ represents a residue of the general formula (II)
R′[CH(OH)—CH(OH)]ₙ II
in which R′ can be an acid group selected from the group consisting of carboxyl, sulfamic acid group, sulfonic acid group, and phosphatc acid group, n denotes a number in the range from 1 to 10,
R² and R³ can be the same or different and represent one of the radicals R¹.

2. 1,3,5-Triazine-2,4,6-tris-hydroxyalkyl acid of claim 1, in which R¹, R² and R³ represent the same radicals of the general formula (II), in which n denotes the number 1 and R′ represents carboxyl group.

3. 1,3,5-Triazine-2,4,6-tris-hydroxyalkyl acid of claim 1, in which R¹, R² and R³ represent the same radicals of the general formula (II), in which n denotes the number 1 and R′ represents sulfamic acid group.

4. A process for the preparation of 1,3,5-Triazine-2,4,6-tris-hydroxyalkyl acid of the general formula (I) 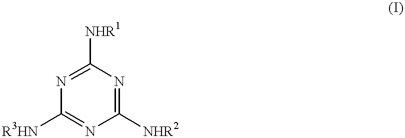
in which R¹ represents a residue of the general formula (II)
R′[CH(OH)—CH(OH)]ₙ (II)
in which R′ can be an acid group selected from the group consisting of carboxyl, sulfamic acid group, sulfonic acid group, and phosphate acid group, n denotes a number in the range from 1 to 10,
R² and R³ can be the same or different and represent one of the radicals R¹, which process comprises: reacting melamine with glyoxal in the presence of acid, the acid being used in a concentration of from 3.0 to 3.5 moles, the glyoxal being used in a concentration of from 3.0 to 4.0 moles, based on 1 mole of melamine.

5. The process of claim 4, in which R¹, R² and R³ represent the same radicals of the general formula (II), in which n denotes the number 1 and R′ represents carboxyl group.

6. The process of claim 4, in which R¹, R² and R³ represent the same radicals of the general formula (II), in which n denotes the number 1 and R′ represents sulfamic acid group.

7. The process of claim 4, wherein the acid is an organic acid selected from the group consisting of glyoxylic acid, formic acid, acetic acid, propionic acid, and citric acid.

8. The process of claim 7, wherein the organic acid is glyoxylic acid.

9. The process of claim 4, wherein the acid is a mineral acid selected from the group consisting of sulfamic acid, sulfuric acid, phosphoric acid, and hydrochloric acid.

10. The process of claim 9, wherein the mineral acid is sulfamic acid.

11. The process of claim 4, wherein the glyoxal is in the concentration of 3.0 moles, the acid is in the concentration of 3.05 moles, based on 1 mole of melamine.
